(19) United States
(12) Patent Application Publication
(10) Pub. No.: US 2007/0238936 A1
(43) Pub. Date: Oct. 11, 2007

(54) Portable Electronic Medical Assistant

(76) Inventor: Shirley Ann Becker, Melbourne Beach, FL (US)

Correspondence Address: IP STRATEGIES, 12 1/2 WALL STREET, SUITE I, ASHEVILLE, NC, 28801, US

(21) Appl. No.: 11/697,960

(22) Filed: Apr. 9, 2007

Related U.S. Application Data
(60) Provisional application No. 60/791,023, filed on Apr. 10, 2006.
(60) Provisional application No. 60/844,287, filed on Sep. 13, 2006.

(51) Int. Cl. A61B 5/00

(52) U.S. Cl. 600/300; 705/2

(57) ABSTRACT

A handheld device used to assist a user in performing daily activities and in being socially active. The device can also be used to assist in monitoring dietary, medication, and supplement intakes, pain and stress levels, vitals, exercise, sleep quality, and other aspects of better living. The device can be personalized to monitor specific diseases or chronic conditions, and can provide access to specific environmental data that can be used to support better living.

### CROSS-REFERENCE TO RELATED APPLICATION

This is related to, and claims the benefit under 35 USC §119(e) of U.S. Provisional Application for Patent No. 60/791,023, which was filed on Apr. 10, 2006, and of U.S. Provisional Application for Patent No. 60/844,287, which was filed on Sep. 13, 2006. The entire disclosures of both of these provisional patent applications are incorporated herein in their entireties.

### FIELD OF THE INVENTION

The invention is related to systems that help users manage tasks associated with daily activities, particularly systems that include a portable device.

### BACKGROUND OF THE INVENTION

Many adults, especially those 50 years and older, need to monitor personal aspects of daily living due to normal aging factors, chronic conditions and illnesses, or disabilities. In addition, they may be homebound due to caregiving responsibilities, lack transportation, or live in rural or remote areas making it more difficult to physically interact with others providing support. Other adults may simply want to be proactive in maintaining or improving aspects of daily living.

### BRIEF SUMMARY OF THE INVENTION

This invention is a portable electronic medical assistant (PEMA) with interface design components and supporting software applications to be used on handheld devices that preferably target middle-aged and older adult users, although it is contemplated that the invention can be used to address medical concerns of any particular individual, regardless of age or condition. The invention promotes better living through aging in place, social interaction, medication and pain management, and proactive healthcare in terms of diet, exercise, and well-being.

The portable electronic medical assistant (PEMA), as described in this document, provides user interface designs for a handheld device (for example, a PocketPC). PEMA also has software applications that support an adult in managing activities; communicating with others; monitoring diet, exercise, and mental health; identifying health patterns associated with environmental factors (for example, weather, pollen, mold, elevation, sunlight, air quality); promoting better living; and being entertained. The PEMA applications support a media-rich user interface for flexibility in using a handheld device anywhere and anytime. PEMA offers unique mobility and portability capabilities for the dynamic monitoring of daily activities such as dietary intake, pain and stress levels, sleep quality, and vital signs, among others. The PEMA applications also offer transparent links to database and Web technologies in building a virtual support network inclusive of friends, family, and healthcare personnel.

PEMA interface designs take into account normal aging factors of vision, cognition, hearing, and physical impairments. These designs promote readability for adults with degrading vision; ease-of-use by those with lost sensitivity in fingertips, stiff finger and hand joints and other motor skill disabilities; intuitiveness for those with cognition issues or early memory loss; and accessibility through the use of sounds, images, and objects for those with impaired vision or hearing. Though the PEMA interface designs target older adult users, they promote universal usability by dealing with the limitations of a handheld device. These limitations include screen glare, small display area, short battery life, and use of a tiny stylus pen required for many commercial applications. As such, anyone can benefit from PEMA's user interface designs and supporting software applications.

PEMA interface designs promote better living through customization and personalization features. PEMA is customizable through the dynamic selection of features such as font size, color scheme, text-to-speech capabilities, as well as button sounds, melodies, and volume control. PEMA is personalized by allowing the user to select aspects of daily living to be monitored or managed. PEMA setup features allow for the selection of zero or more environmental factors (for example, air quality, pollen count, sunlight, wind, precipitation, temperature, and barometric pressure); medications, vitamins, and herbal supplements; and diseases or chronic conditions (for example, asthma, arthritis, Fibromyalgia, diabetes, hypertension, Alzheimer's Disease, depression). Setup features allow for the selection of what is to be tracked regarding dietary intake (for example, calories, trace minerals, vitamin content, trans fat), sleep and stress levels, peak air flow, blood pressure, and pain type, level, and location, among others. The personalization feature supports the generation of reports on the handheld device showing relationships among personal, diet, and environmental data. This report feature provides easy access to correlations of data. The customization and personalization features apply to both a user and a loved one for which care is being provided.

The PEMA interface designs support the capability of electronic interaction through data entry mechanisms. These designs include a screen size qwerty keyboard for typing information content and messages through the use of a fingertip or fat stylus pen. An alternate keyboard design offers even larger keys positioned in alphabetical order and with a separate screen for the numeric keypad to accommodate those with larger fingers and minimal typing skills. A messaging keyboard, with symbols associated with keys, allows for canned text messages to be sent without having to type them. For example, the house symbol located on the keyboard represents the text message “I am home.”

PEMA applications support data transmission transparent to the user thus promoting usability for those with and without technology skills. The user doesn't have to explicitly connect to the Internet; the PEMA applications provide for transparent connectivity. The user may customize the number of scheduled data transmissions occurring within a 24 hour time period. The user can also dynamically transmit data at any time and any place using dial-up, broadband, or wireless Internet connectivity. Data can be stored in the PEMA database until data transmission is completed.

Server software interacts with PEMA applications in retrieving and transmitting data to a centralized database. Data retrieval from a centralized database occurs when the user requests information about diet composition, environmental factors, frequently asked questions, exercise and burned calories, and community resources, among others. Data retrieval is scheduled regularly in order to provide up-to-date information displayed on the handheld device. For example, personalized weather may be downloaded each day to support the user in monitoring his or her health. Data retrieval may also include shared information posted on the user's Web log and stored in the centralized database. A family member, for example, may schedule a doctor's appointment on the shared calendar feature on the Web log. This appointment data is transmitted to the handheld device and displayed on the PEMA calendar. As such, data is transmitted back and forth to the handheld device, stored in its local database, accessed through PEMA and Web interfaces, and supported by PEMA and Web applications.

Daily living data for an individual, stored in a centralized database, may be tracked, managed, and assessed through the user's Web Analyzer component of the Web log for which controlled access is provided to those in the individual's local support network. A set of statistical analysis software tools is made available via the Web Analyzer to provide dynamically-created reports on data entered by an individual as well as reports that provide data correlations among personal, environmental, and other data sources. These Web-based reports provide more detailed information than what is provided by the correlation data displayed on PEMA. For example, a doctor may want to further assess the impact of local changes in the weather and increases in arthritic pain, blood pressure changes and prescription pain medication, or reduced physical activity and changes in mental health. The Web Analyzer component also provides dynamic query capability which further assists in the dynamic evaluation of health trends for better living.

The user can determine the type of data to be shared with members of a support network through the Web Communicator setup feature associated with his or her Web log. Members may include family, friends, healthcare personnel, and others approved by the user. The Web Communicator setup feature of the Web log allows the user to specify membership; as well as data sharing and report capabilities. On a personal level, data mining is supported through the PEMA and Web-based report capabilities. From a broader perspective, data stored in the centralized database provides an opportunity to mine for personal, family, community, regional, and national data trends.

The Web Communicator component provides for bidirectional information exchange with the PEMA. This allows for improved medication management through virtual interaction with pharmacies, clinics, hospitals, and other local network members. The Web Communicator component is accessible to members of the individual's support network for setting up medication and appointment reminders on the PEMA. It also provides a means for accessing information about local, regional, and national resources that can be downloaded on the PEMA.

A community component with data manipulation capabilities promotes better living through the use of integrated technologies with an emphasis on data mining and statistical analysis with personally identifiable information secured or removed. A community may encompass local, state, and federal governments, nonprofit organizations, and commercial industries. Daily living information may be tracked, managed, and assessed from local, regional, national, or international perspectives for better living and improved quality of life. A pharmaceutical company, for example, may want to track the long-term use of an over-the-counter medicine on pain management or the short-term impact of a herbal supplement on weight management, among others.

PEMA supports both a user and a person for whom care is provided. A user may monitor himself or herself in terms of better living; as well as, monitor the health and well-being of a loved one. The portability of a handheld device offers flexibility and mobility in performing caregiving activities anyplace and anytime. The PEMA interface designs and supporting applications offer automated support for scheduling daily events, monitoring health and well being, managing medication and other supplements, socially interacting with others, having available emergency, community, health and family contact information, finding answers to frequently asked questions, as well as being entertained.

### BRIEF DESCRIPTION OF THE DRAWINGS

FIG. 1: Exemplary login screen used to maintain security and privacy of information.

FIG. 2: Exemplary main screen containing main components of PEMA interface.

FIG. 3: Exemplary journal screen containing components for monitoring and managing health and well-being of user and/or person provided care.

FIG. 4: Exemplary Daily Rating screen used to monitor well being of user.

FIG. 5: Exemplary Error Message screen providing feedback on user mistakes or errors.

FIG. 6: Exemplary Emotions screen used to gather feedback on user emotions experienced throughout the day.

FIG. 7: Exemplary Herbal Supplements screen containing personalized list of supplements taken by the user.

FIG. 8: Exemplary Over-the-Counter (OTC) Medications or Supplements screen containing personalized list of OTC taken by the user.

FIG. 9: Exemplary Events screen displaying a personalized list of events.

FIG. 10: Exemplary Results screen displaying user selections.

FIG. 11: Exemplary Behaviors screen containing a personalized list of behaviors that are associated with Alzheimer's Disease.

FIG. 12: Exemplary Qwerty Keyboard screen supporting the use of a fingertip or fat stylus to type a text string.

FIG. 13a: Exemplary Report Generation screen showing a user report associated with user-selected variables.

FIG. 13b: Exemplary environmental data obtained from national and commercial data sources.

FIG. 14: Exemplary Message Keyboard screen containing a keyboard with customized icons representing messages typically sent by the user to one or more contacts.

FIG. 15a: Exemplary Contacts screen supporting user entry of a new contact or the deletion or editing of an existing one.

FIG. 15b: Exemplary New Contact screen used to enter a new contact.

FIG. 16a: Exemplary Medication Management screen used to obtain information about a medication or other supplement.

FIG. 16b: Exemplary Medication Management screen used to view prescription balance, manually update it, or refill the prescription.

FIG. 17a: Exemplary Quick Timer screen supporting the user in setting a quick timer that when activated uses audio and text as part of the PEMA reminder component.

FIG. 17b: Exemplary Quick Timer screen showing quick timers that are activated.

FIG. 17c: Exemplary Quick Timer screen with list of timer titles.

FIG. 18a: Exemplary Calendar screen used to access appointment and event information.

FIG. 18b: Exemplary Scheduling screen whereby the user taps on the checkbox to initiate the scheduling of an appointment or personal event.

FIG. 18c: Exemplary Appointment (or Personal Event) screen gathering data necessary to activate a timer and update the calendar.

FIG. 18d: Exemplary Timer screen specifying the time of an appointment or personal event.

FIG. 18e: Exemplary Appointments screen allowing user to tap on the View button to display appointments and events for a particular day.

FIG. 19a: Exemplary Checklist screen supporting the creation of a checklist using the button concept, as shown in other PEMA interface designs.

FIG. 19b: Exemplary Checklist screen used to support daily activities.

FIG. 20a: Exemplary Customization screen for specifying audio associated with objects, events, and reminders.

FIG. 20b: Exemplary Customization screen offering color scheme options.

FIG. 21: Exemplary Frequently Asked Questions (FAQ) screen providing access to information based on the geographic location of the user.

FIG. 22: Exemplary Pain Management screens whereby the user specifies the body region, identifies pain descriptors, and selects pain level (separate screen not shown).

FIG. 23a: Exemplary PEMA and Information and Communications Technology Schema screen showing a seamless and individualized process of integrating personal data with local and community data sources.

FIG. 23b: Exemplary Data Transmission screen showing alternative PEMA data transmission modes to accommodate users with different computing and ICT skill levels.

FIG. 23c: Exemplary Data Transmission Using Wireless Router screen whereby the communication process is shown for data transmission using a wireless router.

FIG. 24a: Exemplary Bi-Directional Communication screen showing how the Web Communicator component allows for personalized setup of a PEMA in order to fire events associated with managing daily events.

FIG. 24b Exemplary Web Communicator Component screen showing how the PEMA is personalized for use by approved members in the user's support network.

FIG. 25: Exemplary Report Generation Feature screen whereby the PEMA integrated technologies provide access to report features that are accessible by the user and other members (for example, counselor, doctor, or other healthcare personnel).

FIG. 26: Exemplary Web Analyzer Report Generation screen showing how more detailed reports can be accessed via a Web-based component, and how the data gathered on the PEMA is stored in the central database from which data can be mined.

FIG. 27: Exemplary Web Communicator screen illustrates how various databases can be linked to provide personal support for better living.

FIG. 28: Exemplary Architectural View of PEMA and Integrated Technologies screen with three architectural views. The first view is a personal one for managing everyday living. The second view shows PEMA integrated with local data sources (for example, pharmacies, medical records). The third view shows PEMA integrated with national data resources (for example, USDA database of foods and nutritional data, NOAA database with environmental data) and the means to mine personal data for local, regional, and national trends.

### DETAILED DESCRIPTION OF THE INVENTION

### PEMA

PEMA interface designs and applications are made for a handheld device (for example, PocketPC) to assist a user in performing daily activities and being socially active; as well as, assist in monitoring dietary, medication, and supplement intakes, pain and stress levels, vitals, exercise, sleep quality, and other aspects of better living. PEMA may be personalized to monitor specific diseases or chronic conditions. PEMA also provides access to specific environmental data that can be used to support better living. A user with asthma, for example, would have access to air quality, pollen and mold counts, wind speed, and barometric pressure all of which may have an impact on peak air flow.

PEMA interface designs can include very large buttons used to represent objects to be selected by the user (for example, items in a list), a unique navigational schema, customizable color, sound, and font size settings, screen display in landscape mode, graphics and images used to gather information about pain levels and other aspects of daily living, keyboards encompassing the full screen, and informative prompt and message screens all of which support portability and mobility such that the device can be used in various environmental settings (for example, home, work, automobile, clinic, shopping mall, restaurant). In addition, the PEMA applications support a media-rich environment of audio, video, text, voice synthesis, and voice recognition components. PEMA applications can be used in dial-up, wired, or wireless environment through the use of handheld devices.

### PEMA Includes the Following Software Components:

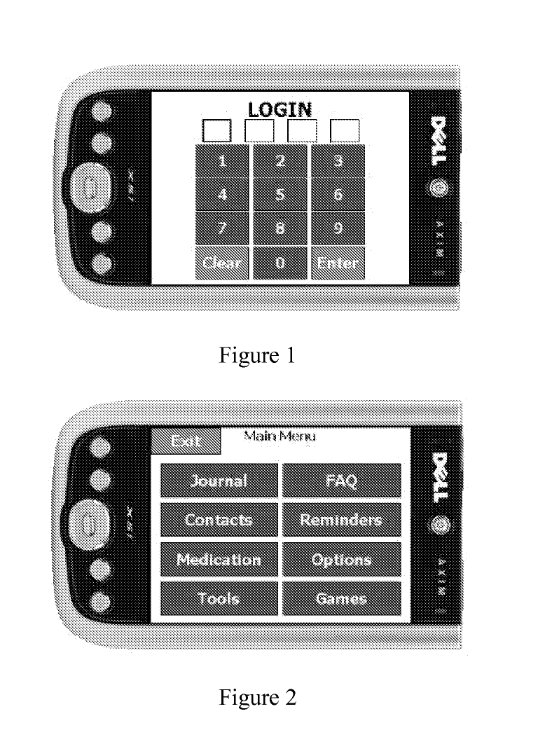

A login secures access to personalized applications and a localized database. FIG. 1 shows a PEMA login screen. The keypad is sufficiently large such that a fingertip or stylus pen can be used for data entry. The keypad supports sounds or melodies that have been selected by the user during setup to be associated with the tapping of a button.

A main menu organizes the personal selections made by the user in monitoring and managing daily life. FIG. 2 illustrates a main menu as part of the PEMA interface. The keys on this and other PEMA interface designs are sized appropriately to support the use of a finger tip or stylus pen for data selection. They may also have audio support when a button or other object is tapped. The main menu components are personalized by the user at the time of PEMA setup.

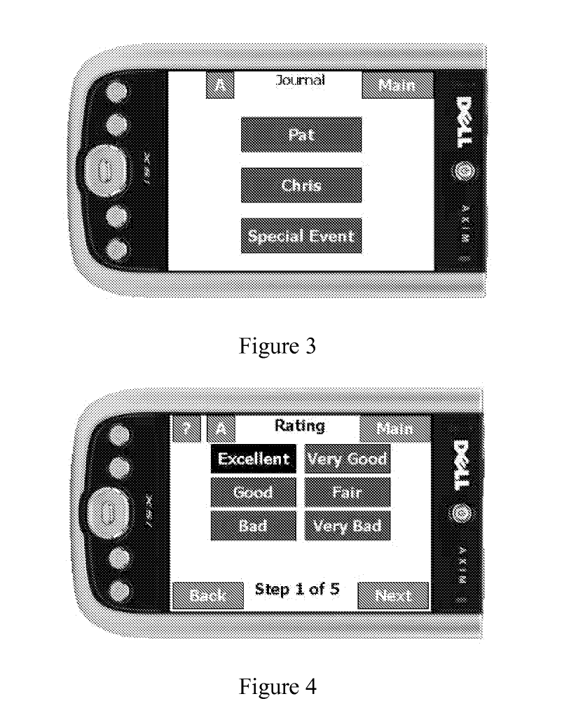

A journal contains components as specified by the user during PEMA setup. FIG. 3 shows a journal with components personalized for Pat (user and caregiver) and Chris (person receiving care). The Special Events button, as a journal component, allows the user to select an event from a predefined list or type in a unique one to be displayed on the Web log. Journal entries will be stored locally in a PEMA database and then transmitted to a centralized source for trend analysis and data mining.

Journal data is typically shared with members of a support network via the Web log to promote social interaction and to identify trends related to health and well-being. It is noted that environmental data can be correlated with personal data to identify factors that have an impact on better living.

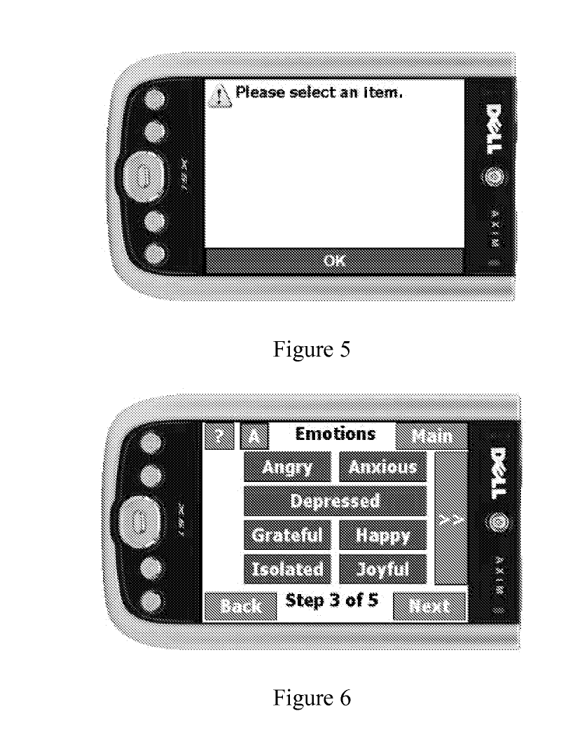

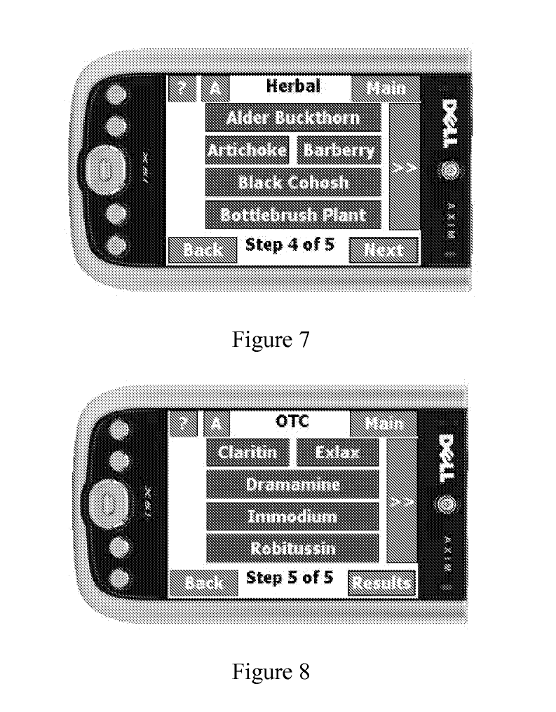

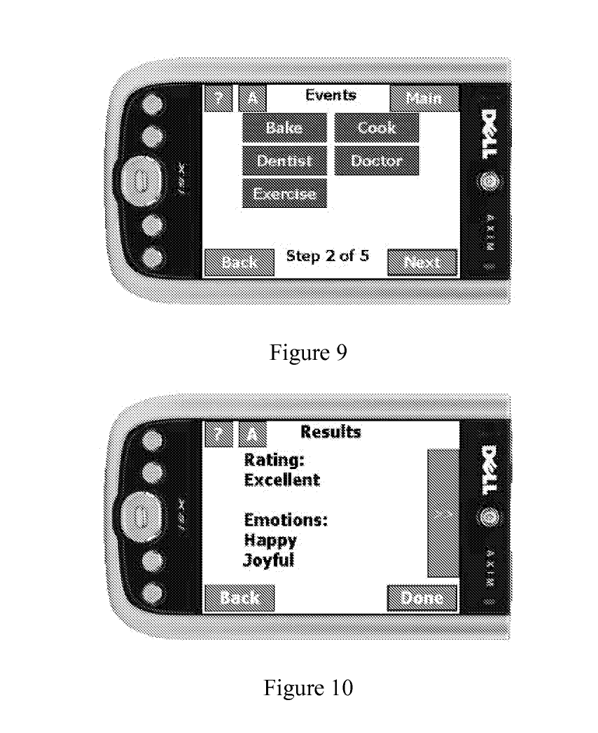

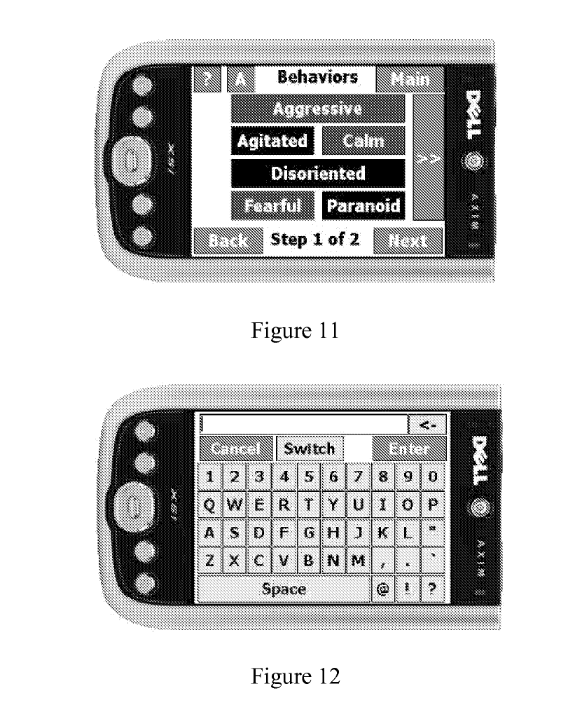

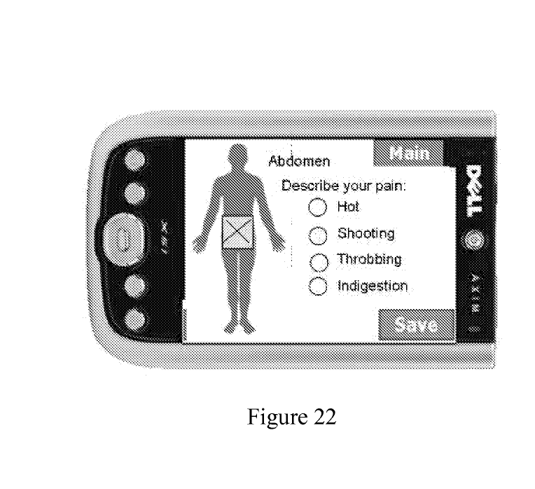

Journal components that offer support for both the caregiver and patient are described below.
Support for User—The PEMA interface designs are personalized through user selection of components made available on the journal screen. Components selected for Pat are further described.
   Daily Rating—FIG. 4 shows a Daily Rating screen. The user taps a button that represents his or her well being for that time period. In this example the highlighted button, Excellent, has been selected by the user.
   Prompts—FIG. 5 shows a message prompt screen used to assist the user in accurate and complete data entries. If the user taps the Next button in FIG. 4 without selecting a daily rating, for example, FIG. 5 would be displayed.
   Emotions—FIG. 6 shows an Emotions screen whereby one or more emotions are selected by the user to represent how his or her day is going.
   Stress—The user selects a stress level on a five point scale to reflect how his or her day is going.
   Vitals—The user enters vital information specific to a disease being monitored. The user may enter peak air flow for asthma, blood sugar level for diabetes, blood pressure numbers for hypertension, etc.
   Sleep Quality—The user enters the number of uninterrupted sleep hours, the total sleep hours, and a rating of sleep quality.
   Pain Level—A graphic image of a human body is displayed on the PEMA screen, as shown in FIG. 22. The user taps a region of the body to enlarge it. This is repeated until an appropriate level of granularity is achieved (for example, fingers are displayed). The user taps a joint, muscle, or internal button to further identify a problem area. The user taps one or more pain description checkboxes (for example, tender, hot, swollen, throbbing) associated with the selected body region. A pain level screen is displayed for which the user taps an appropriate pain level on a five point scale.
Support for User and/or Person Receiving Care—The PEMA interface can be personalized with components selected to support the user and/or components selected to support caregiving activities. The “Pat” journal (user) and/or the “Chris” (person receiving care) journal components may include the PEMA applications described below. Each of these applications would be accessed through the “Pat” or “Chris” button to maintain data integrity.
   Herbal Supplements—FIG. 7 shows an Herbal Supplements screen. The user selects herbal supplements that were taken during the day for himself or herself (or as part of a journal entry for the person receiving care).
   Over the Counter (OTC) Medication—FIG. 8 shows the OTC Medication screen. The user selects OTC medications or supplements that were taken during the day for himself or herself (or as part of a journal entry for the person receiving care).
   Events—FIG. 9 shows an Events screen whereby one or more daily events can be recorded. The user selects one or more events that occurred during the day for either himself or herself (or as part of a journal entry for the person receiving care).
   Results—FIG. 10 shows the Results screen that is displayed at the end of a journal entry. The Results screen allows the user to make changes to data previously entered or save it to the local database for transmission to a centralized database.
Support for Person Receiving Care—The PEMA interface can be personalized to support various components associated with caregiving responsibilities. The following example illustrates the type of support provided.
   Behaviors—FIG. 11 shows the Behaviors screen. The caregiver selects one or more behaviors associated with the one receiving care. In this example, the behaviors relate to Alzheimer's Disease.
Special Events—The user has the option of selecting one or more events from a list (similar in design as the Emotions list presented in FIG. 6). The user also has the option to type an event using one of several screen size keyboards. FIG. 12 shows the qwerty version of the PEMA keyboard that allows the user to type a text string using a fingertip or stylus pen. The user has the option to add typewriter sound when each key is tapped.

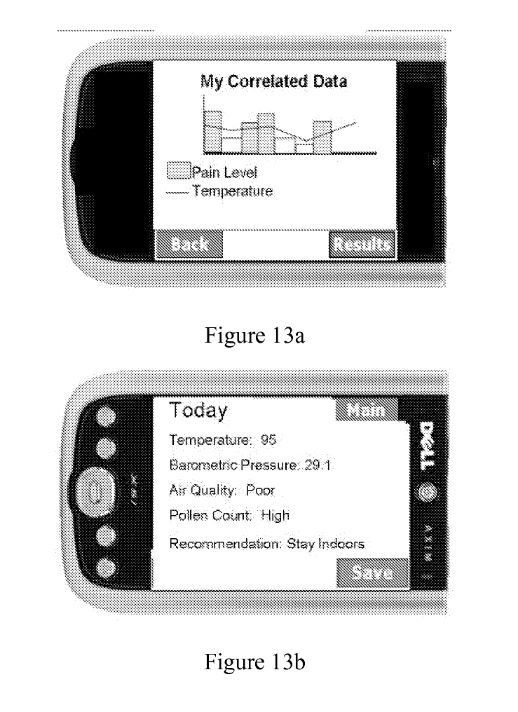

A health trends report generator provides the means to view current and historical data as it relates to other data. As such, correlations of data involving environmental data (for example, pollen count, barometric pressure), dietary composition (for example, amount of Vitamin A), and data entered throughout the day (for example, daily rating of “fair”, pain level of “bad”, sleep quality of “fair”) can be assessed. The user selects the report generation feature during setup and as part of the personalization of PEMA support. FIGS. 13a and 13b illustrate this component.

The data entered into the handheld device through PEMA interface components can be used by family, friends, and healthcare professionals to data mine for long-term health trends. This is made possible through the Web log that shares personal information with members of the user's support network. The stored data contains no personally identifiable information; and as such, the data can be mined by locality, gender, age, disease, diet, weather or other factors that have an impact on better living for an individual, community, or region.

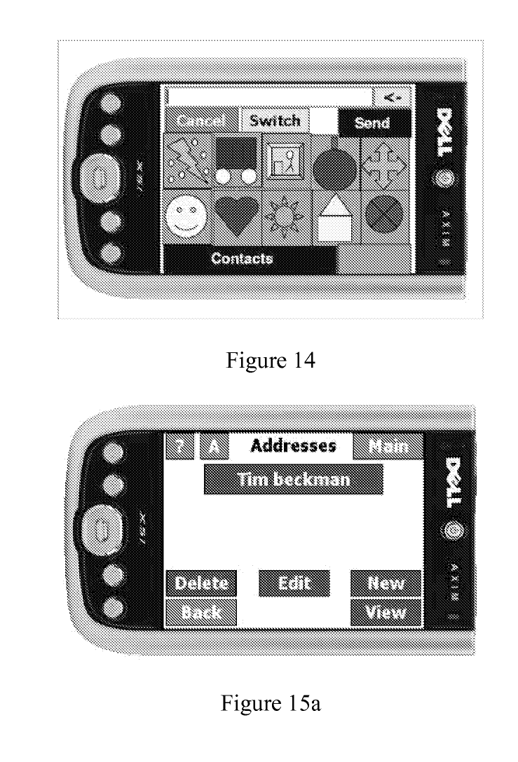

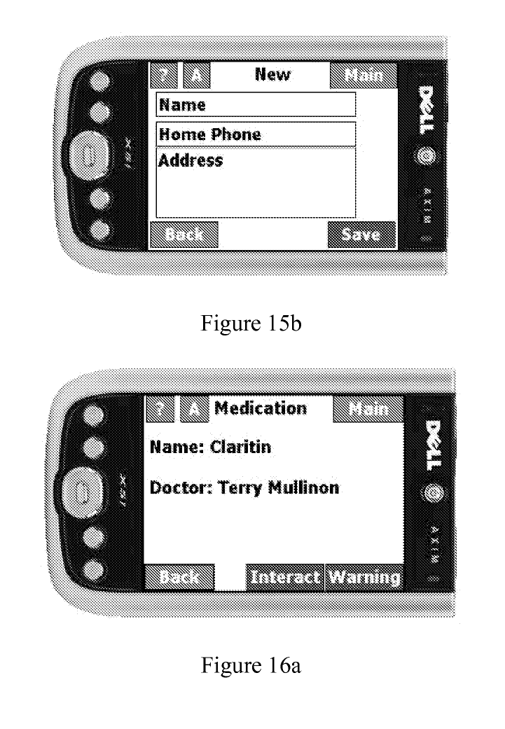

A messaging component supports “quick” text messaging, emergency contact, and email capabilities. The quick text messaging component is supported by a full size keyboard with icons representing canned messages (for example, “call me”, “how are you?”, “I am fine”, “I am home”) that can be sent to email addresses or the Web log. The PEMA interface design, illustrated in FIG. 14, is used to display quick text messages that can be sent by tapping keyboard buttons. FIGS. 15a and 15b show PEMA components for maintaining a list of user contacts that support messaging capabilities.

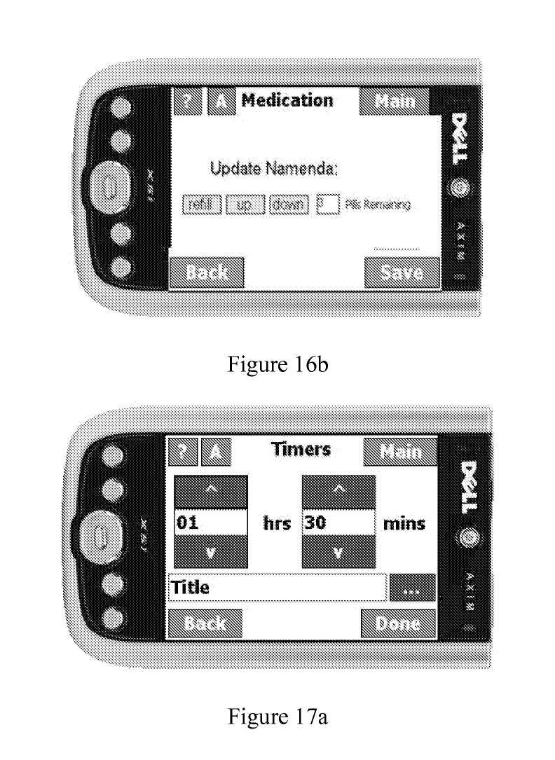

Medication management includes daily reminders, prescription balances, prescription refill reminders, and data access to drug interactions and warnings. (The term “medication” is used to represent prescription drugs, over-the-counter products, vitamins, and other supplements). Reminders can be activated throughout the day to prompt the user to take the medication. The user has the option of responding as to whether the medication was taken or postponing the reminder with activation at a later time. Each prescription is automatically decremented when the user responds to a reminder that the medication was taken. When the prescription reaches a critical level, an automatic reminder is generated for refilling it. The user may select at setup the generation of an automated email to be sent to a pharmacy supporting online prescription refills. FIGS. 16a and 16b illustrates the PEMA components for supporting medication management.

Medication management components are further described below.
Medication Reminder—The medication reminder component automatically provides audio and text messages of medication to be taken by the user or the person for whom care is provided. The user is required to respond to these messages when they appear on the screen. If the user does not respond within specified constraints, the timer is reset and reactivated.
Prescription Refill Reminders—The prescription reminder automatically prompts the user of a low prescription that needs to be refilled.
Prescription Balances—Prescription balance component maintains an inventory that is adjusted when the user responds favorably to taking medication or has had a prescription refilled. After the user responds to a reminder that medication has been administered, the prescription balance is automatically decremented by a predefined amount. (FIG. 16b shows this capability.)
Automatic Update—Through PEMA communication capabilities, there is the potential for a pharmacist, doctor, or other healthcare person to send prescription renewal information to be stored in the local database. The prescription balance may be reset automatically with this information.
Automated Email—An automatic email component provides the capability of notifying the pharmacist (and other interested parties) of a prescription refill and prescription data that has been updated.
Manual Update—The PEMA interface design is such that the user has the capability of reducing or increasing the prescription count by one or more dosages. When the prescription is refilled, the user taps the refill button to adjust the prescription balance.

A calendar supports the scheduling of personal events and appointments such that automated reminders are generated. This component contains timer mechanisms that can be activated to support daily activities.

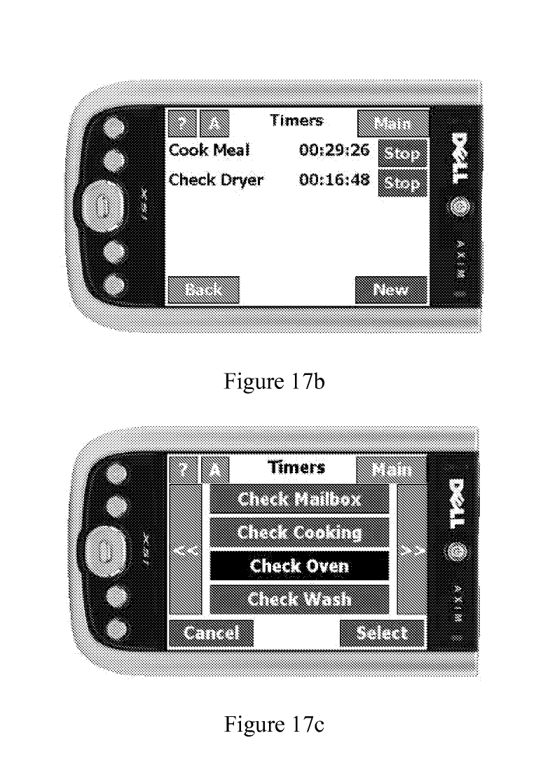

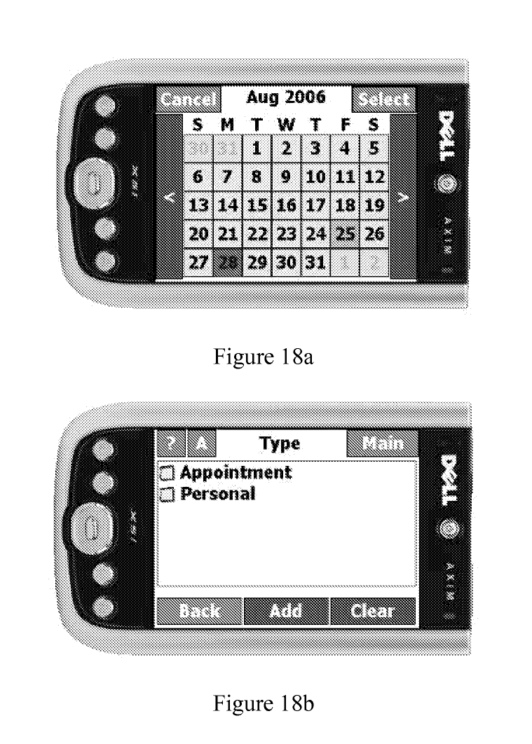

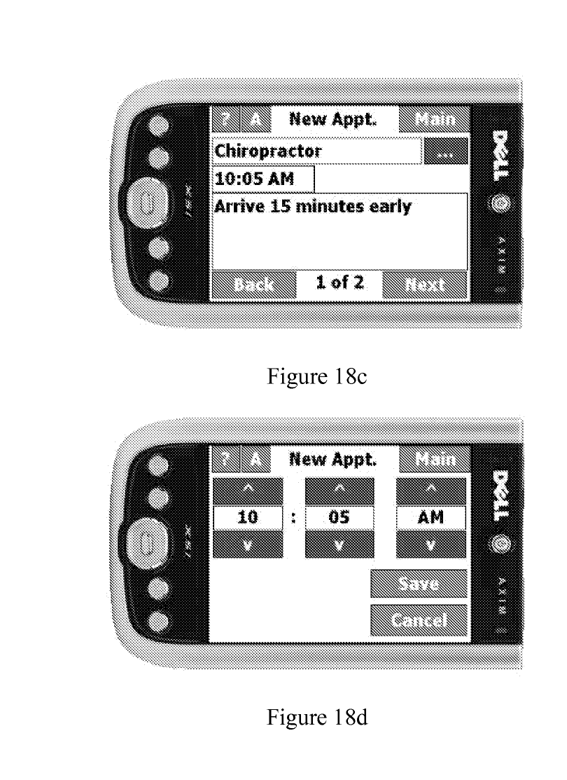

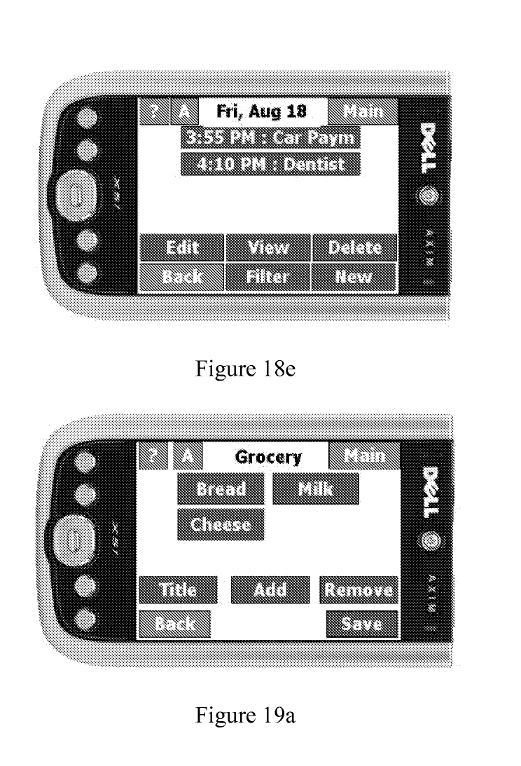

Calendar components are described below.
Quick Timer—FIGS. 17a through 17c show a quick timer component that supports daily tasks. FIG. 17a shows the timer screen whereby the user selects a title from a list by tapping the box with three dots located on the lower right of the screen. FIG. 17c is displayed showing the list of titles. The user may type a unique title by tapping on the Title box located on the bottom of the screen. A keyboard, shown in FIG. 12, is displayed. FIG. 17b shows a screen display of timers that have been set. The user can deactivate a timer by tapping the stop button.
Appointment or Personal Event Scheduler—FIGS. 18a through 18e illustrate a calendar component for scheduling appointments or other personal events. FIG. 18a shows a monthly calendar scrollable by the side navigation bars. FIG. 18b shows a type screen whereby the user initiates the scheduling of an appointment or personal event. FIG. 18c shows the new appointment screen for which the user enters a type, time, and comments that are stored in the local database. FIG. 18d shows the timer screen for specifying the appointment time. This information is used to generate an automated message reminding the user of the appointment. FIG. 18e shows a list of appointments and personal events that have been scheduled. FIG. 18e supports the capability of editing, deleting, or viewing an existing appointment or event or creating a new one. It also supports the capability of filtering the display of either appointments or events.
Synchronization—The calendar component is synchronized with a shared calendar on the user's Web log. Appointments or personal events can be scheduled by members of the user's support network and then downloaded to the PEMA database. The PEMA calendar is updated with this information.

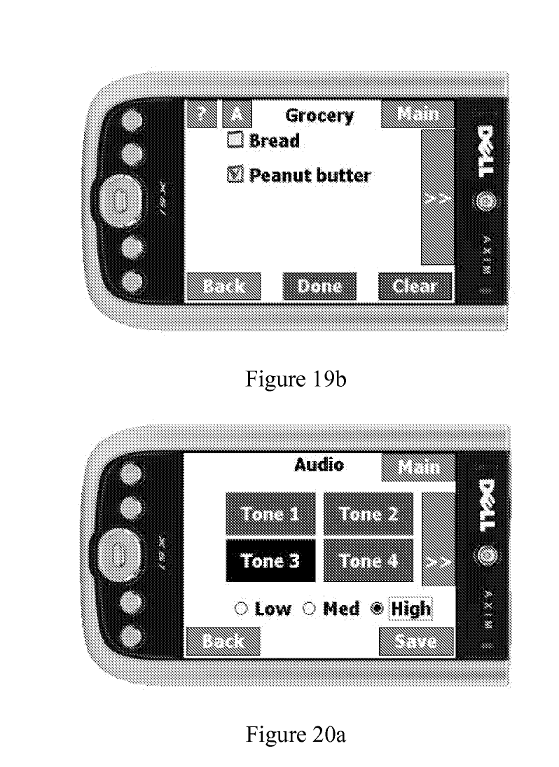

A checklist assists in memory recall of activities to be performed. A checklist can be customized by the user or new ones created for shopping, managing finances, and running errands, among others. In addition, PEMA provides built-in checklists (for example, what to take to adult day care when dropping off a loved one) that can be used with no customization required. FIGS. 19a and 19b show several screens associated with making and using a checklist. FIG. 19a shows several items already created as part of a grocery list. FIG. 19b shows a grocery list being used. The list can be saved for future use or cleared to be reused.

An entertainment feature has games made available to the user to promote memory recall and offer relief for those who are stressed.

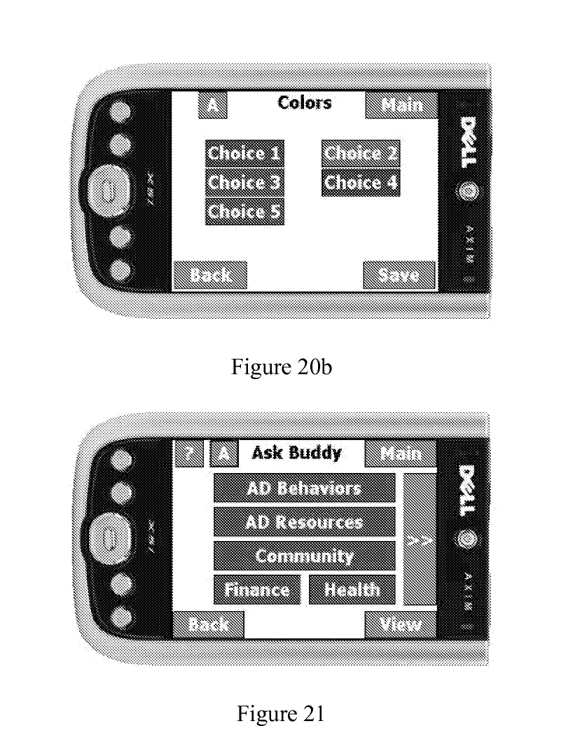

A customization feature allows the user to select an appropriate color scheme that accommodates color deficiency, vision degradation, or vision disabilities. It allows for the selection of voice synthesis such that button labels and other text components are read aloud. It provides the means to select sounds and melodies and adjust their volumes to supplement text reminders, messages, and object selections. It offers the means for dynamic data transmission by tapping a button (for example, Update Now button). FIG. 20a shows the Audio screen for selecting a tone associated with screen objects. FIG. 20b shows a list of color schemes that can be used enhanced readability.

Frequently asked questions (FAQ) component provides access to resources, as shown in FIG. 21. The PEMA setup identifies geographic location in order to gather local, regional, and national resources to be stored on the handheld device and made accessible through the PEMA interface designs.

### Information and Communication Technology (ICT)

PEMA responses to events and reminders as well as user-entered data are stored in a local database to be transmitted to a centralized database. A server component connects with the handheld device such that email, text messages, database entries, and game moves can be communicated to those connected to the network or stored in the server database for data mining capabilities. Security and privacy features are maintained by the server as it communicates with PEMA.

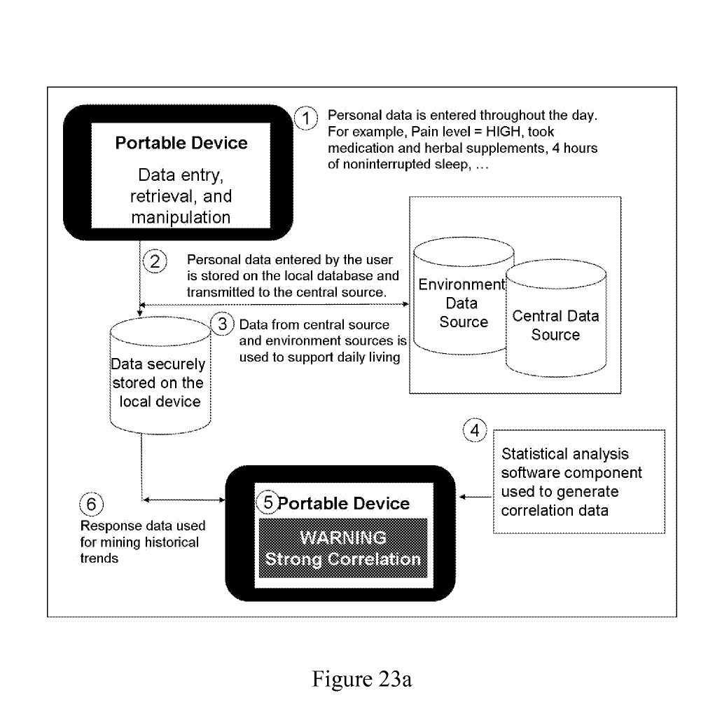

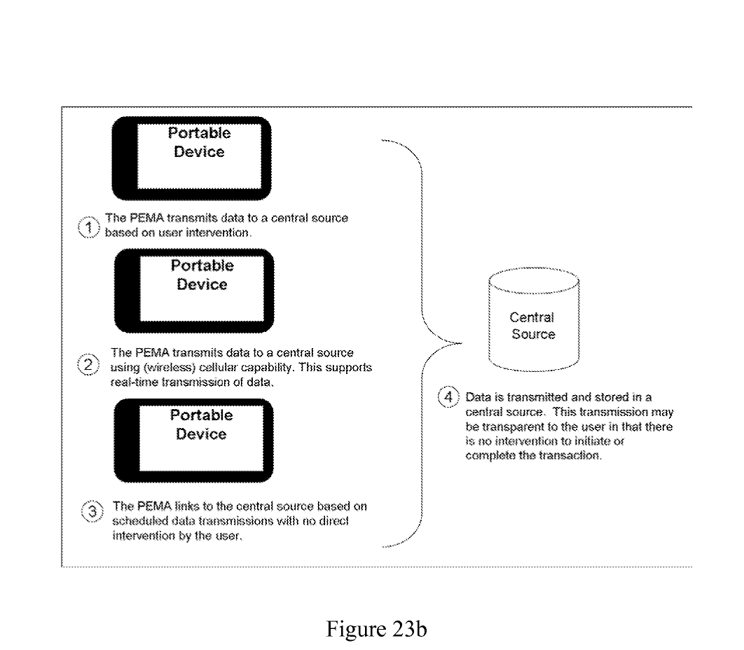

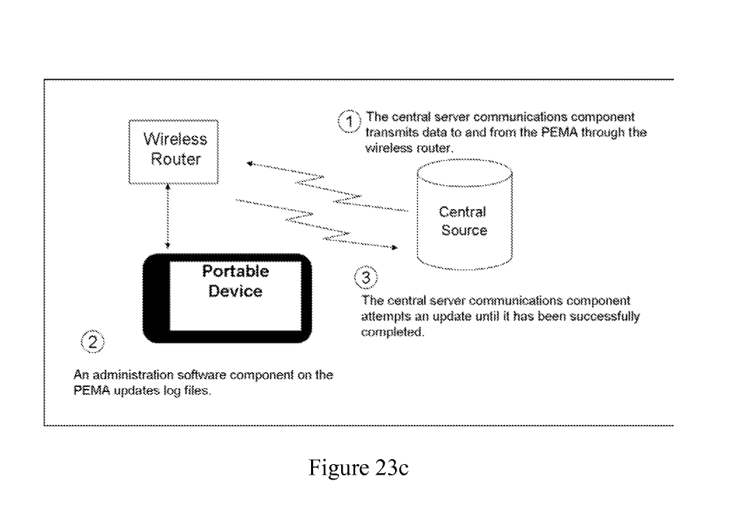

FIG. 23a shows how PEMA and supporting ICT provides a means of promoting better living as a seamless and individualized process. An individual uses PEMA (1) throughout the day to support activities of daily living (ADLs), monitor physical, mental, and emotional health, evaluate diet and exercise choices, promote social interaction, schedule and manage appointments and events, and track usage of medications and supplements, among others. The PEMA allows for on-the-go data entry, retrieval, and manipulation through its unique user interface. The portable device has a local database (2) that stores data in an encrypted format to maintain security and privacy of personally identifiable information. Environment data sources (3) (for example, National Weather Service) and data from a central source (for example, personal historical data) are used to analyze statistically personal data for correlations and confidence levels (though not limited to these). The statistical component (4) utilizes software algorithms to speed performance of complex correlations of data and to pattern match data within and across data sources, among other functions. It also uses statistical data analysis software to perform calculations in real-time in supporting a notification system. A notification system (5), as part of the PEMA, provides statistical feedback in a meaningful and easy to understand format to promote better living. For a person with asthma, for example, seasonal weather patterns and air quality for a particular region are correlated with personal peak air flow capability. This information can be used to manage medication intake, limit types of exercise and outdoor activities, and recommend use of an indoor air purifier, among others.

Data entered by the individual (6) are stored locally and transmitted to a central source to build a historical database with the potential for data mining at individual, local, and community levels. Historical data, entered over time by the individual, can be used by the statistical data analysis software to identify relationships between or among current and historical personal data. Data gathered throughout the day are transmitted to a central data source with the potential to be shared with members of the local network through secured access technology. Data entered by an individual is stored in the PEMA database and transmitted to a central source through real-time data transmissions or regularly scheduled data transmissions.

PEMA and its ICT has a unique feature in transmitting data from the to a central data source. The user does not have to initiate data transmission in order to update the data in the central source and in the device's local database. The use of the technology requires virtually no computing or Internet knowledge or experience. Data can be transparently sent from the device to the central source without any direct intervention of the user.

FIG. 23b shows three scenarios, supported by the PEMA ICT for transmitting data to and from the central source. The first scenario (1) shows the user performing a forced update by using a communications component built into the PEMA software system. The device utilizes either wired or wireless Internet connectivity to complete the forced update. In a home setting, the device can readily be connected to the Internet through existing computing technology (for example, personal computer or laptop wired with Internet access through dial-up or cable, but not limited to these). The second scenario (2) shows data being transmitted through wireless connectivity (for example, cell phone service). This real-time data transmission is the most flexible in maintaining up-to-date information in both the PEMA database and the central source database system. The third scenario (3) represents a home environment whereby there is no Internet connectivity via personal computers, laptops, or other computing devices. The transmission occurs through the use of a wireless router and Internet service (no restrictions on type) made available in the home. The PEMA communications software system transmits the data from the device to the central source. This might happen, for example, when the user is sleeping given there is no direct user intervention required.

FIG. 23c shows the communication process for data transmission directly through a wireless router. The communication component of PEMA (1) allows for data transmission from the local database on the device directly to a central source. In the figure, a wireless router placed in the home allows for transparent communication between the PEMA and the central server communications component. The PEMA (2) maintains log files with information about data transmission. The central communications component (3) attempts to transmit data until it is successful in doing so.

### Supporting Tools and Integrated Technologies

A Web Communicator component allows for the dynamic selection of features and supporting data for the individual use of PEMA. FIG. 23 shows the setup capability of the Web Communicator through the integration of multiple data sources. The individual selects one or more aspects of better living (1) to be supported through the use of PEMA. By selecting a particular disease or health issue, a support system is automatically selected (2). In the example presented in FIG. 23, the user can select from one or more diseases: cancer, stroke (TIA), mental health, diabetes, lower respiratory disease, and/or neuromuscular diseases, among others. If lower respiratory disease is selected, for example, then the daily living component of the PEMA includes the following: exercise, vitals, peak flow, medications, supplements, stress, activities of daily living (ADL), emotions, and daily rating (though not limited to these). The environment data that are automatically selected for inclusion in the portable measurement system include: air quality (for example, pollen, mold, carbon dioxide, smoke, though not limited to these), elevation, and weather (for example, barometric pressure, temperature). Frequently Asked Questions data (3) are customized based on the diseases and/or health issues that have been selected. This information is pulled from the central database as well as local, state, and national resources (but not limited to these). Based on the disease or health issue selected and geographic location, emergency resources are identified. The local members of the support network are also selected (4), though more can be added through this interface at a later date. These members have controlled access to the data gathered via the PEMA as well as the results of correlated data.

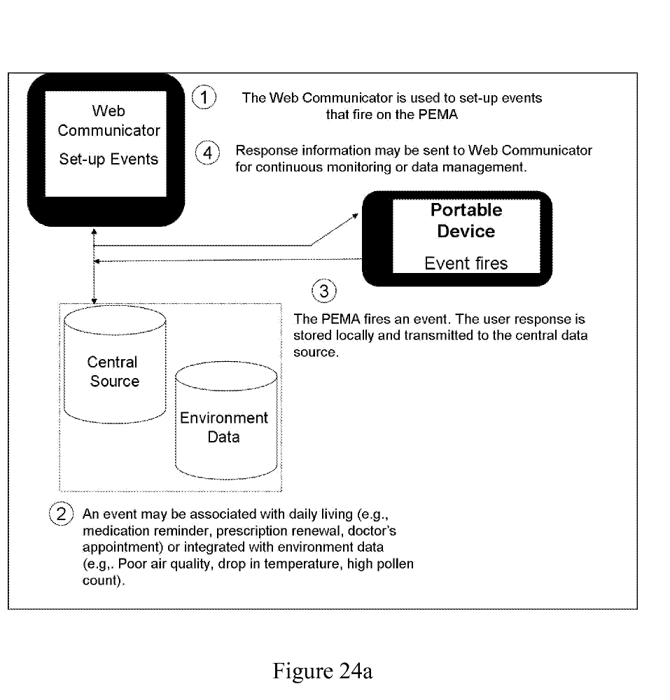

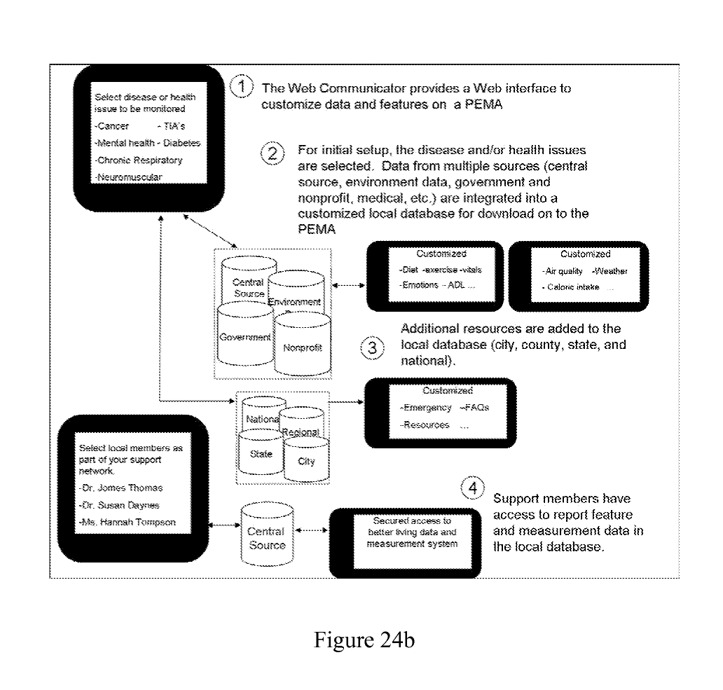

The Web Communicator component customizes controlled access to data entered into a PEMA by an individual. Members of the individual's support network (set up via the Web Communicator Web log tool), can access information directly on the portable device (1) through secured login information provided to them (2). Members may include, but are not limited to, doctors, dentists, physical therapists, dieticians, pharmacists, hospice care personnel, and counselors. FIG. 24 shows this capability of an individual providing an approved member his or PEMA in order to access the report feature. The better living report feature (3) provides access to data trends associated with data entered by the individual (for example, pain levels over time). Correlated data are made available through the report feature on the device such that relationships between or among daily living and environment can be reviewed (4, 5).

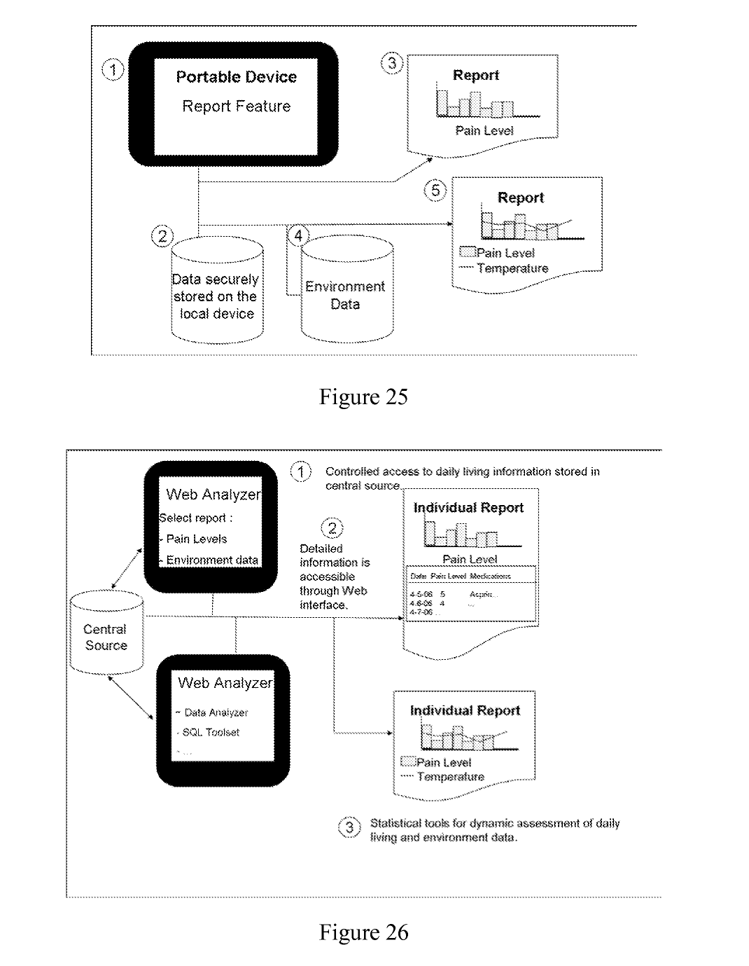

FIG. 25 shows a Web Analyzer tool as part of the user's Web log (1) for more sophisticated data analysis and correlation measurements. These tools allow for more detailed information to be displayed (2), manipulated, and measured (3). The tools can be used to correlate larger data sets, including historical data entered by the individual and external data sources, in order to identify short-term and long-term health trends and identify environment factors impacting better living, among others.

The Web Communicator component also provides the means to electronically link to other systems. A common system is a local pharmacy whereby prescriptions can be filled through electronic means. FIG. 26 shows the Web Communicator component (1) used to initiate a medication management component on the PEMA. The local database is updated with initial prescription amounts (2). Medication reminders, in the form of events firing (3), are scheduled in order to initiate a tracking system on the portable device. A prescription is decremented by the prescribed amount (4) when the individual responds favorably to a reminder. When the prescription balance drops to a specified amount, a refill email (or other means of electronic communication) is sent to the linked pharmacy (4). The pharmacy sends an email note to the PEMA when the prescription is ready for pickup. The prescription balance is reset with the refilled amount (5).

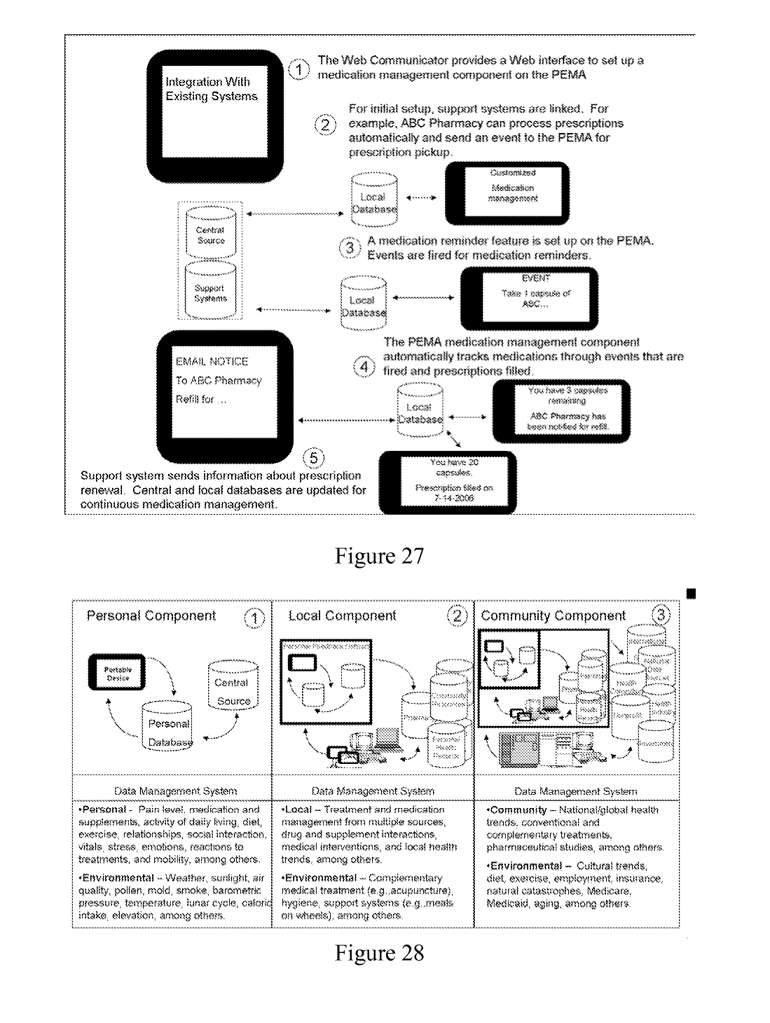

FIG. 27 shows the overall architecture of PEMA and its integrated technologies. The personal component is the user's view of the technology in terms of better living and health management. The personal component (1) manages personal data that is integrated with environmental, local, and community data resources. The local component (2) shows a broader view of PEMA in terms of integration with pharmacies, medical records, community resources, and other data resources. The community component (3) shows an all-encompassing view of PEMA in terms of integration with government, nonprofit, commercial, national, and other data resources. The integration of personal, local, and community data sources provides a wealth of data mining opportunities.

### A Summary of Features Provided by the PEMA Design

According to the invention, any or all of the following features can be provided in any combination, according to the particular application of the invention and the needs and preferences of the individuals involved.
Data Entry Using a Fingertip—PEMA interface components can be manipulated through the use of a finger tip or fat stylus pen in selecting an object, typing text, tapping a button, or navigating through screens. A stylus pen may be used but it is no longer required.
Landscape Mode—PEMA interface components are displayed in landscape mode thus utilizing screen space more efficiently. As a result, objects and text can be enlarged with enhanced usability for those who lack precision capability in the use of a tiny stylus pen. Landscape mode supports the use of the device by both hands such that thumbs or fingers can be used to tap the side navigation bars. It also supports the use of a handheld device in less than ideal settings (for example, low levels of light, screen glare, physical movement).
Navigation Side Bars—The use of enlarged side bars for navigation promotes ease-of-use by those who have shaky hands, inflexible joints, or lost sensitivity in fingertips. It promotes usability for those with eye and hand coordination problems. It also promotes portability when using a handheld device in less than ideal settings. This design supports ease-of-use when scrolling through lists or otherwise maneuvering through screens.
Voice Synthesis—The use of voice synthesis offers multimodal support for those with vision degradation or when using a handheld device in less than ideal settings. The user presses an options button to activate a text-to-voice feature and have the screen contents read aloud.
Dynamic Resizing of Text—The resizing of text is dynamically made available on each screen in order to enhance readability. The user has the capability of resizing text by several font sizes (decrease or increase from the default font size).
Cues—Cues are used on a screen to make PEMA interface designs more intuitive. The “1 of 5 steps” message at the bottom of FIG. 4, for example, is a navigational cue. The cues allow the user to move forward or back through a series of screens without getting lost.
Screen-size Keyboard—Typically, a handheld device requires the use of a built-in, tiny keyboard that is manipulated through the use of a tiny stylus device. This design has been replaced with a screen size keyboard on which content is entered by tapping one or more keys. Data entry can be done using a fingertip, thumb, or stylus pen of any size. The large buttons on the keyboard promote ease-of-use taking into account normal aging factors, disabilities, and usage in less than ideal settings.
Symbol Keyboard—A text message keyboard has a symbol associated with each key in order to send a canned message (for example, “I am home”). This design feature supports text messaging or sending email with little or no typing. The keyboard encompasses the full screen in order to promote usability and to account for usage in less than ideal settings.
Connectivity—The PEMA software applications support transparent Internet connections such that the data in the local database can at anytime be uploaded to and downloaded from a central source. This can be accomplished through dial-up, broadband, or wireless network access. The user has several choices regarding data transmission. The user may send data at any point in time or from any location by pushing an update button as part of the PEMA interface. The user may also select scheduled data transmission times during the setup phase. The second option requires no direct involvement by the user in transmitting data.
Dynamic Lists—Dynamic lists are supported in terms of expanding when items are added or shrinking when items are deleted. Each item may be represented as a button on the screen. For scrolling purposes, navigation bars appear on the screen as the list expands to another screen and disappear when the list shrinks to fit on a single screen.
Buttons as List Items—Large buttons are used to represent items in a list. They have the same functionality as textual descriptions of items that are often displayed in a small font size in handheld applications. However, they are visually easier to read and physically easier to manipulate.
Audio support of objects—The user has an option of adding sound or melodies to PEMA objects. Audio selections can be dynamically changed by the user. The predefined melodies contain notes in appropriate frequency ranges with variations in length in order to accommodate impaired hearing.
Automated reminders—The PEMA allows for automated reminders to be set by the user. The user selects the time by tapping on large buttons. A full screen message prompt and audio sound or melody are generated when the timer is activated.
Calendar feature—A built-in calendar feature offers access to appointments and events that have been scheduled or are to be scheduled. The calendar implements a color code to visually display dates with scheduled appointments or events.
Appointment or event scheduler—The user can schedule a new appointment or event; as well as, change or delete an existing one. An automated timer is associated with each appointment and event and is activated on the scheduled date. These data are made available on the Web log shared calendar and can be updated by members in the user's support network.
Medication management—The user can manage medications through the use of this component. Automated medication reminders are generated for which user responses are stored. These responses may decrement a medication balance for automated refills. The user can setup emails to be sent automatically to pharmacies to place a refill order.
Voice recognition -The PEMA interface supports simple voice commands that will allow the user to activate objects, make selections from a list, or enter data.
Environmental Data Access—Environmental data, such as pollen and mold counts, barometric pressure, temperature, amount of sunlight, and air quality may be downloaded from federal and commercial data sources to be used by PEMA in monitoring health and managing daily activities.
Report Generation—A report generation feature of PEMA allows the user to review compiled data over a period of time. This compilation or correlation may include personal, event-driven, environmental, and other data gathered through PEMA interfaces.
Pain Tracker—A component is the display of a human body that expand into levels of granularity by tapping on the screen. The user taps on PEMA buttons to record information about levels and descriptions of pain associated with the selected body region or part. This data can be used as part of the report generation feature in visually correlating pain data with other personal, event-driven, or environmental data.
Portable technology for virtual access to resources—PEMA provides a virtual link to local, state, national, nonprofit, and commercial resources in order to manage daily activities, diet, medication and supplement intake, and pain levels, among other aspects of daily living.
Portable technology for correlating environment data with personal data—The PEMA, database management system and statistical analysis software components provide comprehensible feedback on correlations between (or among) personal data and environment data.
Portable technology for correlating historical personal data with personal data—The PEMA and statistical analysis software components provide comprehensible feedback on correlations between (or among) historical personal data and current personal data.
Portable technology for managing medication through virtual connections with health resources—The PEMA, communications component, database management system, and Web Communicator provide the means to virtually manage medication, supplements, treatments, and other aspects of daily living. The technology system allows for virtual connectivity and bidirectional communication with pharmacies, clinics, healthcare professionals, and other members of an individual's support network.
Portable technology for sharing daily living information and correlated data with members of a local support network—The PEMA, security software component, database management system, and statistical analysis software components provide quick access to correlated data via a report feature on the portable device.
Web technology for customizing portable device user interface, software applications, statistical data analysis, and virtual connections—The Web Communicator, database management system, communications software component, and PEMA software components provide the means to customize a PEMA in real-time and on a continuous basis. At any time, for example, a disease or health issue can be added as part of the daily living support system residing on the PEMA.
Web technology for statistical report generation using daily living data gathered on a PEMA—The Web Analyzer, communication software components, database management system, and statistical analysis software are used to promote better living health at personal and local levels. A member of an individual's support network has a set of software tools available to analyze daily living information including historical data and correlated environment data.
Data extraction, matching, and integration technology. The data extraction, data matching, and integration toolset along with the statistical analysis software provides the means of correlating data on both PEMA and through its Web Communications and Analyzer components.

I claim:

1. A handheld device to assist a user in performing daily activities and in being socially active.

2. The device of claim 1, adapted to assist in monitoring dietary, medication, and supplement intakes.

3. The device of claim 1, adapted to assist in monitoring pain and stress levels.

4. The device of claim 1, adapted to assist in monitoring vitals.

5. The device of claim 1, adapted to assist in monitoring exercise.

6. The device of claim 1, adapted to assist in monitoring sleep quality.

7. The device of claim 1, personalized to monitor specific diseases or chronic conditions.

8. The device of claim 1, adapted to provide access to specific environmental data that can be used to support better living.

9. The device of claim 8, wherein the specific environmental data includes at least one of air quality, pollen, and mold counts, wind speed, and barometric pressure.
